(19) United States
(12) Patent Application Publication
(10) Pub. No.: US 2004/0072995 A1
(43) Pub. Date: Apr. 15, 2004

(54) Novel fluorescent proteins

(76) Inventors: Sara P. Bjorn, Soborg (DK); Len Pagliaro, Soborg (DK); Ole Thastrup, Soborg (DK)

Correspondence Address: AMERSHAM BIOSCIENCES, PATENT DEPARTMENT, 800 CENTENNIAL AVENUE, PISCATAWAY, NJ, 08855, US

(21) Appl. No.: 10/296,953

(22) PCT Filed: Jun. 18, 2001
(86) PCT No.: PCT/EP01/06848

(30) Foreign Application Priority Data
Jun. 19, 2000 (DK) PA 2000 00953
May 10, 2001 (DK) PA 2001 00739

(51) Int. Cl. C07K 1/00; C07K 14/00; C07K 17/00

(52) U.S. Cl. 530/350

(57) ABSTRACT

A GFP with an F64L mutation and an E222G mutation is provided. This GFP has a bigger Stokes shift compared to other GFPs making it very suitable for high throughput screening due to a better resolution. This GFP also has an excitation maximum between the yellow GFP and the cyan GFP allowing for cleaner band separation when used together with those GFPs.

### FIELD OF INVENTION

The present invention relates to novel variants of the fluorescent protein GFP having improved fluorescence properties.

### BACKGROUND

The discovery that Green Fluorescent Protein (GFP) from the jellyfish A. victoria retains its fluorescent properties when expressed in heterologous cells has provided biological research with a new, unique and powerful tool (Chalfie et al (1994). Science 263:802; Prasher (1995) Trends in Genetics 11:320; WO 95/07463). A very important aspect of using recombinant, fluorescent proteins in studying cellular functions is the non-invasive nature of the assay. This allows detection of cellular events in intact, living cells.

The excitation spectrum of the green fluorescent protein from Aequorea victoria shows two peaks: A major peak at 396 nm, which is in the potentially cell damaging UV range, and a lesser peak at 475 nm, which is in an excitation range that is much less harmful to cells.

To improve the wild type GFP, a range of mutations have been described. Helm (GFP (Helm et al. (1994). Proc. Natl. Acad. Sci. 91:12501) described the discovery of a blue fluorescent variant which has greatly increased the potential applications of using fluorescent recombinant probes to monitor cellular events or functions, since the availability of probes having different excitation and emission spectra permits simultaneous monitoring of more than one process. However, the blue fluorescing variant described by Helm et al, Y66H-GFP, suffers from certain limitations: The blue fluorescence is weak (emission maximum at 448 nm), thus making detection difficult, and necessitating prolonged excitation of cells expressing Y66H-GFP. Moreover, the prolonged period of excitation is damaging to cells especially because the excitation wavelength is in the UV range, 360 nm-390 nm.

Heim et al. (1995), Nature, Vol. 373, p. 663-4, discloses a Ser65Thr mutation of GFP (S65T) having longer wavelengths of excitation and emission, 490 nm and 510 nm, respectively, than the wild-type GFP and wherein the fluorophore formation proceeded about fourfold more rapidly than in the wild-type GFP.

Ehrig et al. (1995) FEBS Letters 367, 163-166, discloses a E222G mutant of the Aequorea green fluorescent protein. This mutation has an excitation maximum of 481 nm and an emission maximum at 506 nm.

Expression of GFP or its fluorescent variants in living cells provides a valuable tool for studying cellular events and it is well known that many cells, including mammalian cells, are incubated at approximately 37° C. in order to secure optimal and/or physiologically relevant growth. Cell lines originating from different organisms or tissues may have different relevant temperatures ranging from about 35° C. for fibroblasts to about 38° C.-39° C. for mouse β-cells. Experience has shown, however, that the fluorescent signal from cells expressing GFP is weak or absent when said cells are incubated at temperatures above room temperature, cf. Webb, C. D. et al., Journal of Bacteriology, October 1995, p. 5906-5911. Ogawa H. et al., Proc. Natl. Acad. Sci. USA, Vol. 92, pp. 11899-11903, December 1995, and Lim et al. J. Biochem. 118, 13-17 (1995). The improved fluorescent variant S65T described by Heim et al. (1995) supra also displays very low fluorescence when incubated under normal culture conditions (37° C.), cf. Kaether and Gerdes FEBS Letters 369 (1995) pp. 267-271. Many experiments involving the study of cell metabolism are dependent on the possibility of incubating the cells at physiologically relevant temperatures, i.e. temperatures at about 37° C.

Thastrup et al. (1997) EP 0 851 874 describes fluorescent proteins that exhibit high fluorescence in cells expressing them when said cells are incubated at a temperature of 30° C. or above. This is obtained with the amino acid in position 1 preceding the chromophore has been mutated. Examples of such mutations are F64L, F64I, F64V F64A and F64G.

Various authors have experimented with combinations of mutations. One such combination is the F64L, S65T GFP (EGFP). EGFP exhibits high fluorescence when expressed at 30° C. or above and has an excitation maximum at 488 nm.

### SUMMARY OF THE INVENTION

The present invention provides novel fluorescent proteins, such as F64L-E222G-GFP that result in a cellular fluorescence far exceeding the cellular fluorescence when expressed at 37° C. and when excitated at 450 to 500 nm compared to the parent proteins, i.e. GFP, the blue variant Y66H-GFP the S65T-GFP variant, and F64L-GFP. This greatly improves the usefulness of fluorescent proteins in studying cellular functions in living cells.

It is shown that GFP mutated at the 64 position from F to L (F64L) and at the 222 position from E to G (E222G) has remarkable properties. It is first shown that the F64L,E222G-GFP has an entirely different spectrum than F64L,S65T-GFP (Example 2). In contrast, there is no substantial difference between folding characteristics (measured as the time when fluorescence is observed between the two GFPs, Example 3). Likewise, there was no difference between the pH sensitivity of the two GFPs (Example 4). The observed brightness of the E222G versus the S65T mutated F64L-GFPs is dependent on the test conditions (Example 5).

### DETAILED DESCRIPTION OF THE INVENTION

One aspect of the present invention relates to a fluorescent protein derived from Green Fluorescent Protein (GFP) or any functional GFP analogue, wherein the amino acid in position 1 preceding the chromophore has been mutated and wherein the Glutamic acid in position 222 has been mutated said mutated GFP has an excitation maximum at a higher wavelength compared to F64L-GFP and the fluorescence is increased when the mutated GFP is expressed in cells incubated at a temperature of 30° C. or above compared to wildtype GFP.

The excitation and emission characteristics of the F64L,E222G-GFP differ significantly from wild-type GFP and EGFP. Existing fluorescent proteins have demonstrated utility for research applications such as quantitative fluorescence microscopy (Patterson, G. H., et al (1997). Biophysical J. 73:2782-2790; Piston, D. W., et al (1999) Meth. Cell Biol. 58:31-48). It is now clear, however, that the optimal fluorescent protein characteristics for high-throughput screening (HTS) applications in drug discovery differ somewhat from those for research applications (Kain, S. R. (1999) Drug Discovery Today 4:304-312). For example, factors such as optimal and signal/noise are more important for HTS applications in drug discovery than are absolute brightness of probes such as fluorescent proteins. The F64L,E222G-GFP described in this patent application has an excitation maximum of 470 nm and an emission maximum of 505 nm (see FIG. 3:), compared to the respective excitation and emission maxima of 490 nm and 510 nm for EGFP. This results in a Stokes shift of 35 nm for F64L,E222G-GFP, as compared to 20 nm for EGFP. This results in a significant increase in the excitation-emission band separation for F64L,E222G-GFP relative to EGFP with several implications for the use of F64L,E222G-GFP in high-throughput screening. Some of these are listed below:

1. The increased Stokes shift of F64L,E222G-GFP results in increased spectral resolution of its excitation and emission peaks. This enables more complete band separation using a conventional dichroic beam-splitter, and decreased background signal for assays incorporating F64L,E222G-GFP relative to assays based on EGFP.

2. F64L,E222G-GFP fluorescence can be excited by conventional light sources using narrow band filters, or commercially available laser producing lines at 472 nm. In either case, the greater Stokes shift of F64L,E222G-GFP results in lower cross-talk from excitation light to the toe of the emission spectrum.

3. The excitation maximum of F64L,E222G-GFP falls midway between those of the cyan fluorescent protein variant (ECFP, excitation max ˜433 nm) and the yellow fluorescent protein variant (EYFP, excitation max ˜513 nm). Because of this, it will allow for cleaner band separation when used together with those probes, and it is optimized for assay applications in which several GFP-labeled components will be multiplexed.

Many sources of GFPs exist. Examples are GFP derived from Aequorea victoria and GFP derived from Renilla. Various GFPs have been isolated from Renilla examples are reniformis and mulleri. As described in the examples and in SEQ ID NOS: 3 and 4, the chromophore in Aequorea victoria is in position 65-67 of the predicted primary amino acid sequence of GFP. Thus, in a preferred embodiment the GFP is derived from Aequorea victoria.

It is preferred that the mutation at F64 is a mutation to an aliphatic amino acid. Examples are F64L, F64I, F64V, F64A, and F64G, wherein the F64L substitution being most preferred. However other mutations, e.g. deletions, insertions, or post-translational modifications immediately preceding the chromophore are also included in the invention, provided that they result in improved fluorescence properties of the various fluorescent proteins. It should be noted that extensive deletions may result in loss of the fluorescent properties of GFP.

The E222G, E222A, E222V, E222L, E222I, E222F, E222S, E222T, E222N, E222Q substitutions are preferred, the E222G substitution (that is substitution to Glycine) being most preferred.

A preferred sequence of the gene encoding GFP derived from Aequorea victoria is disclosed in SEQ ID NO: 3 (enhanced) and in SEQ ID NO: 7 (jelly fish). SEQ ID NO: 1 shows the nucleotide sequence of F64L-GFP with humanised codon. SEQ ID NO: 5 shows the nucleotide sequence of F64L-GFP with jellyfish codon. Besides, the novel fluorescent proteins may also be derived from other fluorescent proteins as mentioned above.

Herein the abbreviations used for the amino acids are those stated in J. Biol. Chem. 243 (1968), 3558.

One aspect of the invention relates to a nucleotide sequence coding for the Fluorescent protein F64L-E222G-GFP. An example of such F64L-E222G-GFP is shown in list 2. In a preferred aspect the nucleotide sequence is in the form of a DNA sequence.

The DNA construct of the invention encoding the novel fluorescent proteins may be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA construct may also be prepared by polymerase chain reaction (PCR) using specific primers, for instance as described in U.S. Pat. No. 4,683,202 or Saiki et al., Science 239 (1988), 487-491. A more recent review of PCR methods may be found in PCR Protocols, 1990, Academic Press, San Diego, Calif., USA.

The DNA construct of the invention may be inserted into a recombinant vector which may be any vector which may conveniently be subjected to recombinant DNA procedures. The choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the fluorescent protein of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the fluorescent protein of the invention.

The promoter may be any DNA sequence which shows transcriptional activity in the host cell of choice and may be derived from genes encoding proteins either homologous or heterologous to the host cell, including native Aequorea GFP genes.

Examples of suitable promoters for directing the transcription of the DNA sequence encoding the fluorescent protein of the invention in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1(1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpIA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehel lipase, A. oryzae alkaline protease, A. oryzae triose phosphate isomerase or A. nidulans acetamidase. Preferred are the TAKA-amylase and gluA promoters.

Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gene, or the Bacillus pumilus xylosidase gene, or by the phage Lambda P_{R} or P_{L} promoters or the E. coli lac, trp or tac promoters.

The DNA sequence encoding the novel fluorescent proteins of the invention may also, if necessary, be operably connected to a suitable terminator, such as the human growth hormone terminator (Palmiter et al., op cit.) or (for fungal hosts) the TPI1 (Alber and Kawasaki, op. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, .g. ampicillin, kanamycin, tetracyclin, chloramphenicol, neomycin or hygromycin. For filamentous fungi, selectable markers include amdS, pyrG, argB, nlaD, sC.

The procedures used to ligate the DNA sequences coding for the fluorescent protein of the invention, the promoter and optionally the terminator and/or secretory signal sequence, respectively, and to insert them into suitable vectors containing the information necessary for replication, are well known to persons skilled in the art (cf., for instance, Sambrook et al., op. cit.).

The host cell into which the DNA construct or the recombinant vector of the invention is introduced may be any cell which is capable of expressing the present DNA construct and includes bacteria, yeast, fungi and higher eukaryotic cells.

Examples of bacterial host cells which, on cultivation, are capable of expressing the DNA construct of the invention are grampositive bacteria, e.g. strains of Bacillus, such as B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gramnegative bacteria such as Echerichia coli. The transformation of the bacteria may be effected by protoplast transformation or by using competent cells in a manner known per se (cf. Sambrook et al., supra).

Examples of suitable mammalian cell lines are the HEK293 and the HeLa cell lines, primary cells, and the COS (e.g. ATCC CRL 1650), BHK (e.g. ATCC CRL 1632, ATCC CCL 10), CHL (e.g. ATCC CCL39) or CHO (e.g. ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad, Sci. USA 79 (1982), 422-426; Wigler et al., Cell 14 (1978), 725; Corsaro and Pearson, Somatic Cell Genetics 7 (1981), 603, Graham and van der Eb, Virology 52 (1.973), 456; and Neumann et al., EMBO J. 1 (1982), 841-845.

Examples of suitable yeast cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding the fluorescent protein of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 230 023, EP 184 438.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

One aspect of the invention relates to a host transformed with a DNA construct according to any of the preceding aspects. The transformed or transfected host cell described above is then cultured in a suitable nutrient medium under conditions permitting the expression of the present DNA construct after which the cells may be used in the screening method of the invention. Alternatively, the cells may be disrupted after which cell extracts and/or supernatants may be analysed for fluorescence.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection).

In the method of the invention, the fluorescence of cells transformed or transfected with the DNA construct of the invention may suitably be measured in a spectrometer or a fluorescence microscope where the spectral properties of the cells in liquid culture may be determined as scans of light excitation and emission.

One aspect of the invention relates to a fusion compound consisting of a fluorescent protein (F64L-E222G-GFP ), wherein the (F64L-E222G-GFP ) is linked to a polypeptide. Examples of such polypeptide is kinase, preferably the catalytic subunit of protein kinase A, or protein kinase C, or Erk1, or a cytoskeletal element.

The invention further relates to a process for preparing a polypeptide, comprising cultivating a host according to any of the preceding aspects and obtaining therefrom the polypeptide expressed by said nucleotide sequence.

The various aspects of the invention have a plethora of uses. Some of these are described below:

Use of F64L-E222G-GFP in an in vitro assay for measuring protein kinase activity, or dephosphorylation activity, or for measuring protein redistribution.

Use of F64L-E222G-GFP as a protein tag in living and fixed cells. Due to the strong fluorescence the novel proteins are suitable tags for proteins present at low concentrations. Since no substrate is needed and visualisation of the cells does not damage the cells dynamic analysis can be performed.

Use as an organelle tag. More than one organelle can be tagged and visualised simultaneously in living cells, e.g. the endoplasmic reticulum and the cytoskeleton.

Use as a secretion marker. By fusion of F64L-E222G-GFP to a signal peptide or a peptide to be secreted, secretion may be followed on-line in living cells. A precondition for that is that the maturation of a detectable number of novel fluorescent protein molecules occurs faster than the secretion.

Use as genetic reporter or protein tag in transgenic animals. Due to the strong fluorescence of the novel proteins, they are suitable as tags for proteins and gene expression, since the signal to noise ratio is significantly improved over the prior art proteins, such as wild-type GFP.

Use as a cell or organelle integrity marker. By co-expressing two of the novel proteins, the one targeted to an organelle and the other expressed in the cytosol, it is possible to calculate the relative leakage of the cytosolic protein and use that as a measure of cell integrety.

Use as a marker for changes in cell morphology. Expression of the novel proteins in cells allows easy detection of changes in cell morphology, e.g. blebbing, caused by cytotoxic agents or apoptosis. Such morphological changes are difficult to visualize in intact cells without the use of fluorescent probes.

Use as a transfection marker, and as a marker to be used in combination with FACS sorting. Due to the increased brightness of the novel proteins the quality of cell detection and sorting can be significantly improved.

Use as real-time probe working at near physiological concentrations. Since F64L-E222G-GFP is significantly brighter than wild type GFP and F64L-GFP when expressed in cells at about 37° C. and excited with light at about 490 nm, the concentration needed for visualization can be lowered. Target sites for enzymes engineered into the novel proteins, e.g. F64L-E222G-GFP, can therefore be present in the cell at low concentrations in living cells. This is important for two reasons: 1) The probe must interfere as little as possible with the intracellular process being studied; 2) the translational and transcriptional apparatus should be stressed minimally.

The novel proteins can be used as reporters to monitor live/dead biomass of organisms, such as fungi. By constitutive expression of F64L-E222G-GFP in fungi the viable biomass will light up.

Transposon vector mutagenesis can be performed using the novel proteins as markers in transcriptional and translational fusions.

Transposons to be used in microorganisms encoding the novel proteins. The transposons may be constructed for translational and transcriptional fusions. To be used for screening for promoters.

Transposon vectors encoding the novel proteins, such as F64L-E222G-GFP, can be used for tagging plasmids and chromosomes.

Use as a reporter for bacterial detection by introducing the novel proteins into the genome of bacteriophages.

By engineering the novel proteins, e.g. F64L-E222G-GFP, into the genome of a phage a diagnostic tool can be designed. F64L-E222G-GFP will be expressed only upon transfection of the genome into a living host. The host specificity is defined by the bacteriophage.

The invention is further illustrated in the following examples with reference to the appended sequence lists.

TABLE 1
List of sequences

| Name | Nucleotide SEQ ID NO: | Protein SEQ ID NO: |
|---|---|---|
| e-F64L-GFP | 1 | 2 |
| (PS399) | | |
| e-F64L-E222G-GFP | 3 | 4 |
| (PS699) | | |
| jf-F64L-GFP | 5 | 6 |
| (PS350) | | |
| jf-F64L-E222G-GFP | 7 | 8 |
| (PS1186) | | |

### LEGEND TO FIGURES

PS codes are explained in Table 2.

FIG. 1: Excitation spectra of PS1189 (excitation maximum at 492 nm), PS1191 (excitation maximum at 468 nm), PS1185 (excitation maximum at 490 nm) and PS1186 (excitation maximum at 473 nm). The emissions were recorded at 560 nm. The samples of PS1189 and PS1191 were 2-fold diluted and the samples of PS1185 and PS1186 were 10-fold diluted.

FIG. 2: Emission spectra of PS1189 (emission maximum at 509 nm), PS1191 (emission maximum at 505 nm), PS1185 (emission maximum at 510 nm) and PS1186 (emission maximum at 506 nm). Excitation was at 430 nm. The samples of PS1189, PS1191 and PS1185 were 2-fold diluted and the sample of PS1186 was 10-fold diluted. The curves for PS1189 and PS1191 relate to the primary y-axis whereas the curves for PS1185 and PS1186 relate to the secondary y-axis.

FIG. 3: Overlapping excitation (Ex) and emission (Em) spectra of PS1189 (panel A), PS1191 (panel B), PS1185 (panel C), and PS1186 (panel D). The excitation curve to the left and the excitation curve to the right relate to the primary and secondary y-axis, respectively.

FIG. 4 This figure shows the images collected after Lipofectamine 2000 transfection. eF64L,E222G (PS699) is at the top of the right column referred to as E222G, eF64L,S65T-GFP (PS279) is at the top of the left column referred to as EGFP.

FIG. 5 Comparing the pH sensitivity of EGFP (PS279) and eF64L,E222G-GFP (PS699).

### EXAMPLES

### Example 1

Construction of GFP Plasmids

Plasmids pEGFP-N1 (GenBank accession number U55762) and pEGFP-C1 (GenBank accession number U55763) both contain a derivative of GFP in which one extra amino acid has been added at position two to provide a better translational start sequence (a Kozak sequence) and so the total number of amino acids is increased by one to 239 instead of the 238 found in wildtype GFP. Therefore the denomination of mutations in GFP in these plasmids strictly should be referred to as e.g. F65L rather than F64L. However, to avoid this source of confusion and because the GFP community has adopted the numbering system of wildtype GFP in its communications, the numbers used here conform to the commonly used naming of mutations in wildtype GFP. The relevant mutations in this respect are F64L, S65T, and E222G.

Plasmids pEGFP-N1 and pEGFP-C1 contain the following mutations in the chromophore: F64L and S65T. The codon usage of the GFP DNA sequence has been optimized for expression in mammalian cells. N1 and C1 refer to the position of multiple cloning sites relative to the GFP sequence.

To construct a plasmid combining F64L and E222G, pEGFP-N1 and pEGFP-C1 were first subjected to PCR with primers 9859 and 9860 described below. The primers are complementary to the DNA sequence around the chromophore region and introduce a point mutation changing the threonine at position 65 to serine. In addition the primers introduce a unique Spe1 restriction site by silent mutation. The 4.7 kb PCR products were digested with Spe1, religated, and transformed into E. coli. The resulting plasmids are referred to as PS399 (N1 context) and PS401 (C1 context). These plasmids contain the chromophore sequence 64-LSYG-67. Plasmids PS399 and PS401 were subjected to Quick-Change mutagenesis (Stratagene) employing PCR with primers 0225 and 0226 described below. These primers are complementary to sequences near the C-terminus of the GFP and change glutamate at position 222 to glycine, and in addition they introduce an Avr2 restriction site by silent mutation. The resulting plasmids are referred to as PS699 (N1 context) and PS701 (C1 context). They combine an LSYG chromophore with E222G with humanised codon and is referred to as eF64L,E222G (see sequence list 2).

9859-top:
5′-TGTACTAGTGACCACCCTGTCTTACGGCGTGCA-3′
9860-bottom:
5′-CTGACTAGTGTGGGCCAGGGCACGGGCAGC-3′
0225-bottom:
5′-CCCGGCGGCGGTCACGAACCCTAGGAGGACCATGTGATCGCG-3′
0226-top:
5′-CGCGATCACATGGTCCTCCTAGGGTTCGTGACCGCCGCCGGG-3′

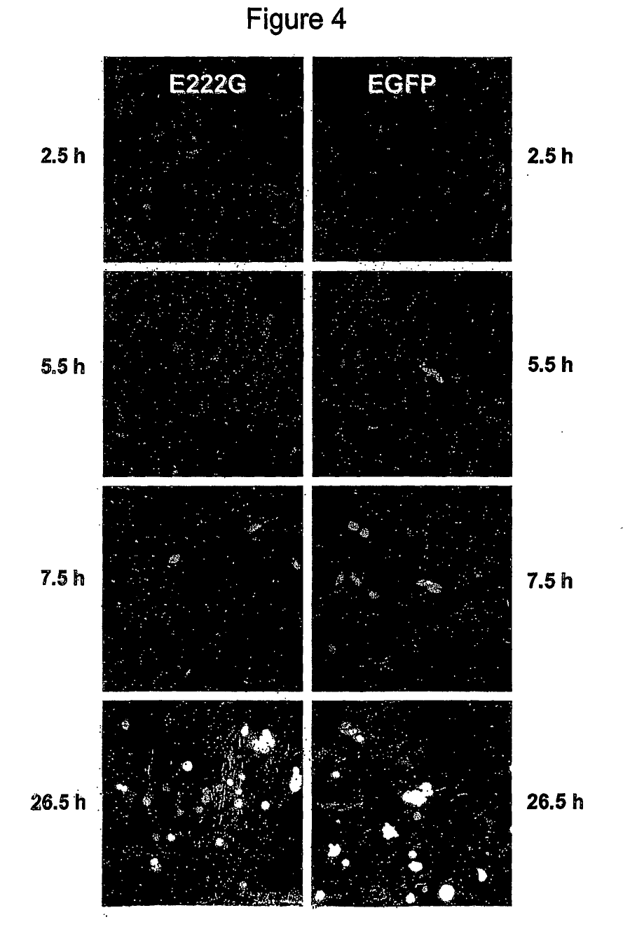

A plasmid encoding a GFP directly derived from jellyfish with F64L (disclosed in FIG. 4 of WO97/11094,) was subjected to PCR with primers 9840 & 9841 described below. The PCR product was digested with restriction enzymes Age1 and Acc65 and ligated into pEGFP-N1 digested with Age1 and BsrG1. This replaces EGFP with F64L-GFP and introduces an amino acid change L236G near the c-terminus as a consequence of joining Acc65 and BsrG1 sites. This plasmid is referred to as PS350.

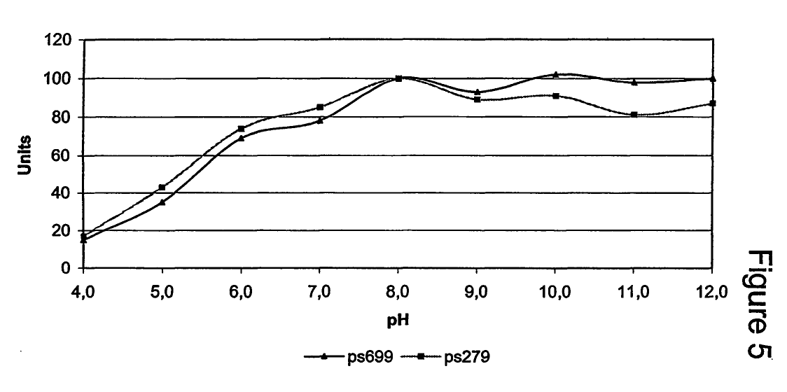

A plasmid encoding a GFP directly derived from jellyfish with F64L, S65T (disclosed in FIG. 5 of WO97/11094,) was subjected to PCR with primers 9840 & 9841 described below. The PCR product was digested with restriction enzymes Age1 and Acc65 and ligated into pEGFP-N1 digested with Age1 and BsrG1. This replaces EGFP with F64L, S65T-GFP and introduces an amino acid change L236G near the c-terminus as a consequence of joining Acc65 and BsrG1 sites. This plasmid is referred to as PS351.

Plasmid PS350 was subjected to QuickChange PCR (Stratagene) with primers 0317 & 0318 described below. This introduces E222G by mutation and an Avr2 restriction site by silent mutation. This plasmid is referred to as PS832.

Plasmid PS832 was subjected to QuickChange PCR (Stratagene) with primers 0325 & 0326 described below. This introduces L64F by mutation and a Psp1406 restriction site by silent mutation. This plasmid is referred to as PS845.

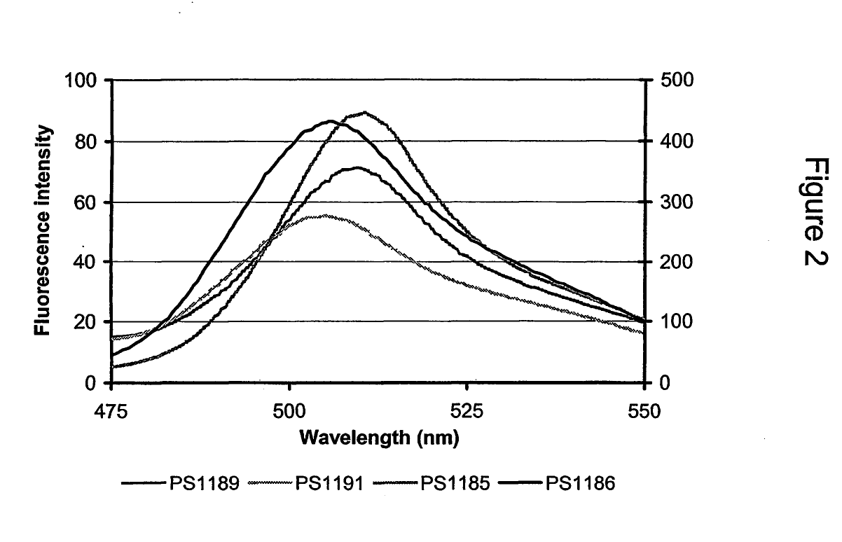

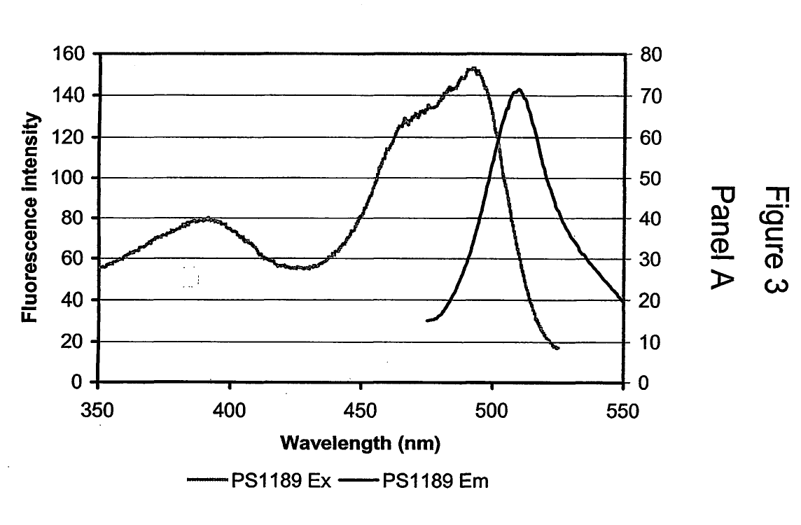

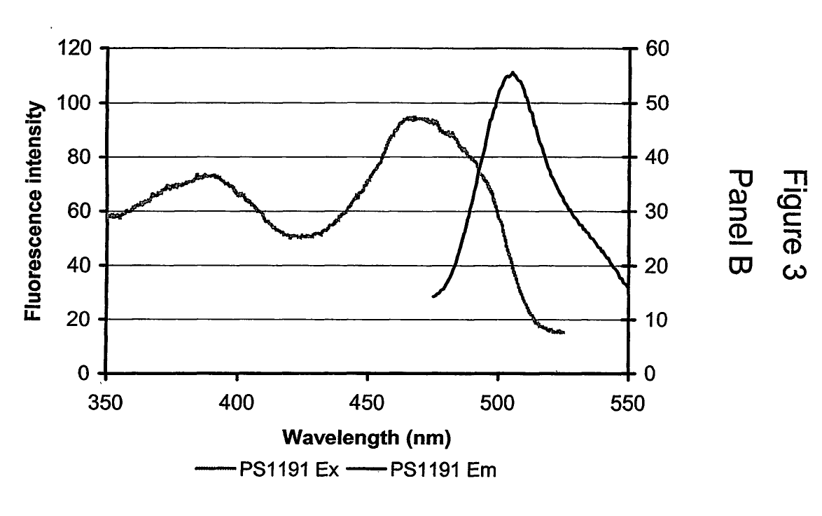

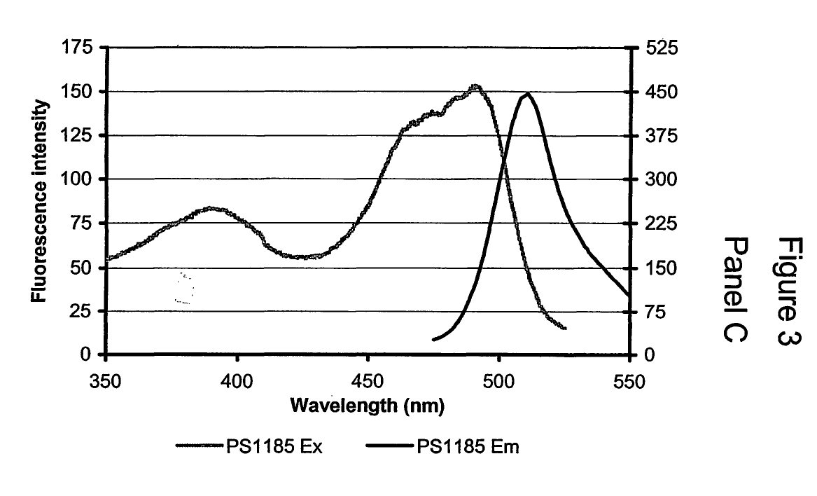

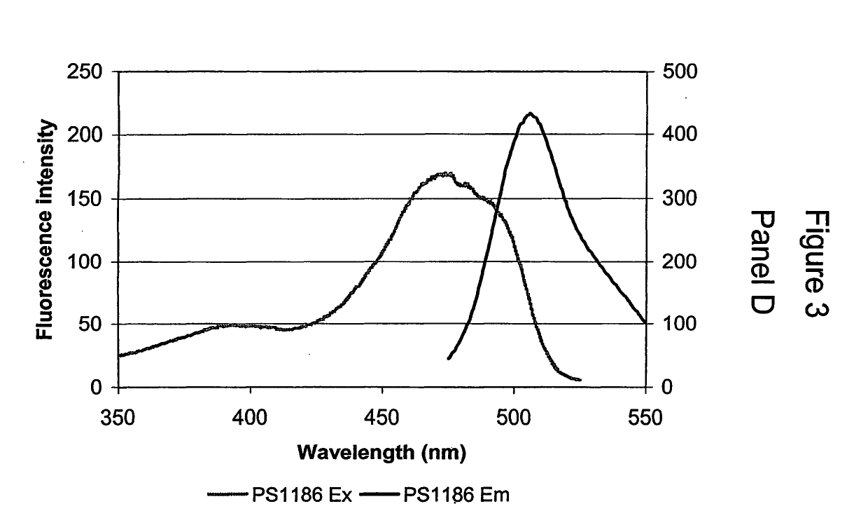

A plasmid encoding a GFP directly derived from jellyfish (disclosed in FIG. 2a of WO97/11094) was subjected to PCR with primers 9840 & 9841 described below. The PCR product was digested with restriction enzymes Age1 and Acc65 and ligated into pEGFP-N1 digested with Age1 and BsrG1. This replaces EGFP with wildtype GFP and introduces an amino acid change L236G near the c-terminus as a consequence of joining Acc65 and BsrG1 sites. This plasmid is referred to as PS854.

Plasmid PS399 was subjected to QuickChange PCR (Stratagene) with primers 0327 & 0328 described below. This introduces L64F by mutation and a Psp1406 restriction site by silent mutation. This plasmid is referred to as PS844.

Plasmid PS699 was subjected to QuickChange PCR (Stratagene) with primers 0327 & 0328 described below. This introduces L64F by mutation and a Psp1406 restriction site by silent mutation. This plasmid is referred to as PS846.

9840-top:
5′-GTACCGGTCACCATGAGTAAAGGAGAAGAAC-3′
9841-bottom:
5′-TTATTGGTACCCTTCATCCATGCCATGTG-3′
0317-top:
5′-GAGATCACATGATCCTCCTAGGGTTTGTAACAGCTGCTGGG-3′
0318-bottom:
5′-CCCAGCAGCTGTTACAAACCCTAGGAGGATCATGTGATCTC-3′
0325-top:
5′-CCAACGCTTGTCACAACGTTTTCTTATGGTGTTC-3′
0326-bottom:
5′-GAACACCATAAGAAAACGTTGTGACAAGCGTTGG-3′
0327-top:
5′-CCCACACTAGTGACAACGTTTTCTTACGGCGTGC-3′
0328-bottom:
5′-GCACGCCGTAAGAAAACGTTGTCACTAGTGTGGG-3′

Plasmids encoding GFPs in jellyfish codon context (PS350, PS351, PS832, PS845, PS854) were subjected to PCR with primers 1259 and 1260 described below. The ca 0.8 kb PCR products were cut with restriction enzymes BspH1 and BamH1, and ligated into E. coli expression vector pTrcHis (from Invitrogen) cut with Nco1 and BamH1. This places the GFPs under control of the ITPG-inducible promoter in the vector. The bottom primer 1260 also changes the glycine at position 236 back to leucine. The resulting plasmids are referred to as P81184 (jf-F64L-GFP), PS1185 (jf-F64L,S65T-GFP), PS1186 (jf-F64L,E222G-GFP), PS1187 (jf-E222G-GFP) and PS (jf-GFP).

Plasmids encoding GFPs in humanised enhanced codon context (PS279=pEGFP-N1 (Clontech), PS399, PS699, PS844, P8846) were subjected to PCR with primers 1261 and 1262 described below. The ca 0.8 kb PCR products were cut with restriction enzymes Nco1 and BamH1, and ligated into E. coli expression vector pTrcHis (from Invitrogen) cut with Nco1 and BamH1. This places the GFPs under control of the ITPG-inducible promoter in the vector. The resulting plasmids are referred to as PS1189 (e-F64L,S65T-GFP=EGFP), PS1190 (e-F64L-GFP), PS1191 (e-F64L,E222G-GFP), PS1192 (e-GFP) and PS1193 (e-E222G-GFP).

1259-top:
5′-GTTGTTTCATGAGTAAAGGAGMGAACTTTTC-3′
1260-bottom:
5′-GTTGGATCCTTATTTGTATAGTTCATCCATG-3′
1261-top.
5′-GTTGTTCCATGGTGAGCAAGGGCGAGGAGCTG-3′
1262-bottom:
5′-GTTGGATCCTTACTTGTACAGCTCGTCCATG-3′

The plasmids described above were transformed into E. coli strain DH5alpha (Life Technologies). Single colonies were picked and grown overnight at 37C. In LB medium containing 1 mM IPTG. 0.5 ml cells were pelleted and stored at −20C. until they were analyzed.

TABLE 2
Summary table of plasmids encoding GFPs with indicated amino acids
at positions 64, 65 and 222.

| mammalian | | Backbone- | aa | aa | aa | E. coil |
|---|---|---|---|---|---|---|
| cell expres- | | codon | pos | pos | pos | expression |
| sion plasmid | | usage | 64 | 65 | 222 | plasmid |
| PS846 | e-E222G-GFP | enhanced | F | S | G | PS1193 |
| PS844 | e-GFP | enhanced | F | S | E | PS1192 |
| PS699 | e-F64L, | enhanced | L | S | G | PS1191 |
| | E222G-GFP | | | | | |
| PS399 | e-F64L-GFP | enhanced | L | S | E | PS1190 |
| PS279 | EGFP | enhanced | L | T | E | PS1189 |
| PS854 | jf-GFP | jellyfish | F | S | E | PS1188 |
| PS845 | jf-E222G-GFP | jellyfish | F | S | G | PS1187 |
| PS832 | jf-F64L, | jellyfish | L | S | G | PS1186 |
| | E222G-GFP | | | | | |
| PS351 | jf-F64L, | jellyfish | L | T | E | PS1185 |
| | S65T-GFP | | | | | |
| PS350 | jf-F64L-GFP | jellyfish | L | S | E | PS1184 |

### Example 2

Determination of Spectral Properties of Proteins EGFP and eF64L,E222G.

Plasmids expressing EGFP from plasmid pEGFP-N1 (also referred to as PS279), and eF64L,E222G from plasmid PS699 were transfected into E. Coli TOP10 cells (Invitrogen) using lipofectamine 2000 (from Life Technologies) according to manufacturers recommendations. After 5 days cells were collected and resuspended in extraction buffer 50 mM TRIS (pH8.0) with 1 mM DTT. Cells were lysed by 3 cycles of freeze-thaw. Cell debris was centrifuged out at 10000 g in acooled centrifuge. NaCl was added to 100 mM.

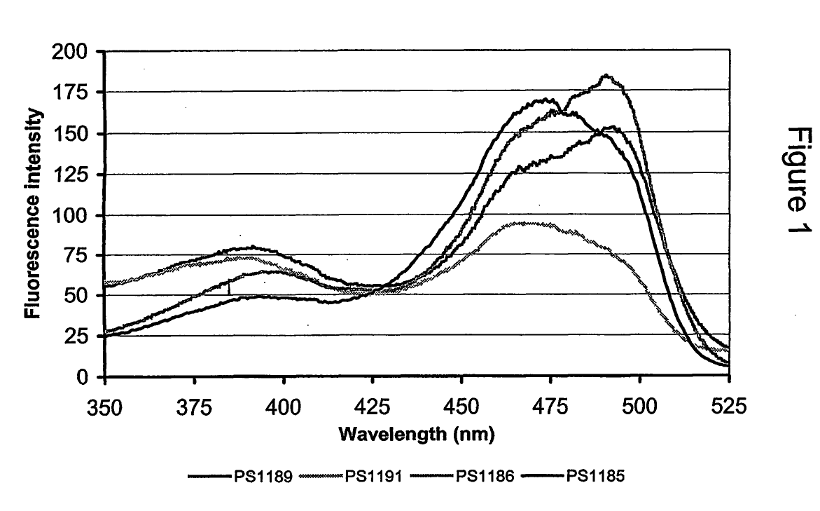

The cell pellets were resuspended in 1000 μl of H₂O each (2-fold dilution relative to volumes of pelleted cultures) and transferred to 1.0×0.5 cm plastic cuvettes and the following excitation and emission spectra were recorded on a Perkin Elmer LS50B luminescence spectrometer:

| | |
|---|---|
| Excitation spectrum: | |
| Excitation at 350-525 nm | Emission 560 nm (10 nm slit width) |
| (5 nm slit width) | |
| Data presented in FIG. 1. | |
| Emission spectrum: | |
| Excitation at 430 nm | Emission 450-550 nm (5 nm slit width) |
| (10 nm slit width) | |
| Data presented in FIG. 2. | |

Using the same settings, excitation and emission spectra of 10-fold (200 μl of 2-fold diluted cells mixed with 800 μl of water) diluted cells were recorded for the strongly fluorescent samples expressed from cDNAs with jellyfish backbone (PS1185 and PS1186).

In contrast to the expression levels, the fluorescence properties of the probes were independent of the codon usage. The spectra recorded for the probes with Thr65:E222 (PS1185 and PS1189) were very similar (excitation and emission maxima at 490-492 nm and 509-510 nm, respectively) and with Stokes shifts of 17-20 nm. Likewise, the spectra recorded for the probes with Ser65:G222 (PS1186 and PS1191) were very similar (excitation and emission maxima at 468-473 nm and 505-506 nm, respectively) and with Stokes shifts of 33-37 nm.

### Example 3

Determination of Time to Fluorescence of EGFP and eF64L,E222G in CHO Cells.

Three, 2 well chambers with CHOhIR cells were transfected with plasmid PS279 expressing EGFP and plasmid PS699 expression eF64L,E222G using the Lipofectamine transfection method.

Fluorescence from the cells was checked at regular intervals after transfection.

Lipofectamine 2000 transfection method was used to transfect EGFP and F64L,E222G in one, 8-w II chamber with CHOhIR cells. Fluorescence from the cells was check d at regular intervals after transfection as described above. Images were taken from the sam cell fields at each interval. Three different fields were observed for each plasmid. The microscope and camera settings were the same for each image. Optimal exposure time was taken from a chamber of cells with full EGFP expression (transfected 24 hours previously) to ensure the exposure does not saturate. The first images were taken from 45 minutes to 1 hour post transfection, thereafter with a 30-minute interval for the first 7.5 hours post transfection and an image was collected 26.5 hours post transfection. Five different fields were observed for each plasmid. Fluorescence was detected no later then 4 hours post transfection. Fluorescence in eF64L,E222G was detected in one field 2.5 hours post transfection. In the remaining fields, fluorescence was detected no later than 4 hours post transfection (FIG. 4).

### Example 4

Comparing pH Sensitivity Over Range pH 4.0 to pH 12.0 Between EGFP and eF64L,E222G.

Samples of semi-purified EGFP from PS279 and eF64L,E222G from PS699 proteins produced in COS7 cell expression are tested for pH sensitivity over a range from pH 4.0 to pH 12.5, with 0.5 point intervals. Excitation and emission scans were taken of each protein at the pH values of 4.0, 8.0, and 12.5. The results of the scans found EGFP's excitation max to be 490 nm and emission max to be 510 nm and eF64L,E222G's excitation max to be 475 nm and emission max to be 504 nm. Different pH values did not affect the excitation or emission max. Single reads were made with excitation at 470 nm, emission at 510 nm and with 10 nm slits. The results show no clear differences between EGFP and eF64L,E222G regarding pH sensitivity, except what could be due to random fluctuation (FIG. 5). This experiment has been repeated with essentially same result.

### Example 5

Comparison of Relative Brightness of GFPs.

10 plasmids were constructed which combine some of the following features:

F or L at position 64.

S or T at position 65.

E or G at position 222.

“jellyfish” or “humanised enhanced” GFP backbone.

The plasmids were transfected into CHO cells. One, two and four days later the cells were inspected visually in a fluorescence microscope by two people. The excitation was 475/40=blue light and the emission 510-560=green light. Cells were scored on a “green” scale ranging from essentially black to extremely bright (Table 3). Results did not change much with time.

TABLE 3

| | | GFP | codon | aa | aa | aa |
|---|---|---|---|---|---|---|
| Plasmid | “greenness” | (* UVmax) | context | 64 | 65 | 222 |
| PS854 | black | Jf-GFP * | jellyfish | F | S | E |
| PS845 | almost black | jf-GFP-E222G | jellyfish | F | S | G |
| PS846 | almost black | e-GFP-E222G | humanised | F | S | G |
| PS844 | almost black | e-GFP * | humanised | F | S | E |
| PS350 | light green | jf-GFP-F64L * | jellyfish | L | S | E |
| PS351 | green | jf-GFP-S65T | jellyfish | L | T | E |
| PS832 | green | jf-GFP-F64L, | jellyfish | L | S | G |
| | | E222G | | | | |
| PS399 | bright green | e-GFP-F64L * | humanised | L | S | E |
| PS699 | very bright green | e-GFP-F64L, | humanised | L | S | G |
| | | E222G | | | | |
| PS279 | very bright green | EGFP | humanised | L | T | E |

The plasmids were also transfected into HeLa cells. After 24 hours transfection the cells were run on a FACS Calibur flow cytometer for characterisation of whole cell fluorescence, with excitation at 488 nm and emission viewed with fluorescence filter set 530/30 nm (range 515-545 nm). 10000 events were collected for each transfection and 2 replicates carried out for each construct. Average fluorescent intensities from the FACS analysis were obtained as geometric means (mean fluorescence on log scale) and results are shown in Table 4.

TABLE 4

| | | GFP | codon | aa | aa | aa |
|---|---|---|---|---|---|---|
| Plasmid | FACS | (* UVmax) | context | 64 | 65 | 222 |
| PS845 | 5.4 | jf-GFP-E222G | jellyfish | F | S | G |
| PS854 | 5.5 | jf-wtGFP * | jellyfish | F | S | E |
| PS350 | 9.3 | if-BioGreen * | jellyfish | L | S | E |
| PS846 | 9.4 | e-wtGFP-E222G | humanised | F | S | G |
| PS832 | 16.5 | jf-BioE222G | jellyfish | L | S | G |
| PS351 | 22.2 | if-BioST | jellyfish | L | T | E |
| PS844 | 24.5 | e-wtGFP * | humanised | F | S | E |
| PS399 | 73.3 | e-BioGreen * | humanised | L | S | E |
| PS699 | 209.2 | e-BioE222G | humanised | L | S | G |
| PS279 | 421 | EGFP | humanised | L | T | E |

It is clear from the table above that, when expressed in the mammalian HeLa cell, the GFPs with humanised codon are far brighter than the GFPs with jellyfish codon. EGFP and e-BioE222G being the brightest. It is no surprise that EGFP is about twice as bright as e-BioE222G under these conditions. The excitation at the FACS is at 488 nm, close the excitation maximum of EGFP at 490 nm. As illustrated in Table 5 below 97% of the emission from EGFP will be picked up, whereas only 86% from the e-BioE222G. Furthermore, the difference between the intensity of EGFP and e-bioE222G when excited at the e-bioE222G excitation maximum of 470 is not as pronounced.

TABLE 5

| | PS1189 | PS1191 | PS1185 | PS1186 |
|---|---|---|---|---|
| | eLTE | eLSG | jfLTE | jfLSG |
| Emission intensity with excitation | 131.4 | 94.1 | 155.0 | 167.2 |
| at 470 nm | | | | |
| Emission intensity with excitation | 148.1 | 80.4 | 178.2 | 151.2 |
| at 488 nm | | | | |
| Excitation max | 492 nm | 468 nm | 490 nm | 473 nm |
| Emission intensity at excitation | 152.9 | 93.8 | 183.3 | 169.1 |
| max | | | | |
| Ratio: Em. intensity(488)/Em. | 0.97 | 0.86 | 0.97 | 0.89 |
| intensity(max) | | | | |
| Emission max | 509 nm | 505 nm | 510 nm | 506 nm |
| Emission intensity at emission max | 71.2 | 55.6 | 444 | 432 |

In mammalian cells nhanced GFPs were brighter than jellyfish GFPs. In E. Coli. jellyfish GFPs were brighter than enhanced GFPs. Thus, when it is worthwhile to choose the GFP backbone with care according to the subsequent host.

1. A fluorescent protein derived from Green Fluorescent Protein (GFP) or any functional GFP analogue, wherein the amino acid in position 1 preceding the chromophore has been mutated and wherein the Glutamic add in position 222 has been mutated said mutated GFP has an excitation maximum at a higher wavelength and the fluorescence is increased when the mutated GFP is expressed in cells incubated at a temperature of 30° C. or above compared to wild-type GFP.

2. A fluorescent protein according to the preceding claim, wherein the chromophore is in position 65-57 of the predicted primary amino acid sequence of GFP.

3. A fluorescent protein according to any one of the preceding claims, said protein being derived from Aequoria victorea or Renilla.

4. A fluorescent protein according to any one of the preceding claims, wherein the amino add F in position 64 of the GFP has been substituted by an aliphatic amino acid.

5. A fluorescent protein according to any one of the preceding claims, wherein the amino add F in position 64 of the GFP has been substituted by an amino acid selected from the group consisting of L, I, V, A and G.

6. A fluorescent protein according to any one of the preceding claims, wherein the amino acid F in position 64 of the GFP has been substituted by L.

7. A fluorescent protein according to any one of the preceding claims, wherein the amino acid E in position 222 of the GFP has been substituted by an amino add selected from the group consisting of G, A, V, L, I, F, S, T, N, and Q.

8. A fluorescent protein according to any one of the preceding claims, wherein the amino acid E in position 222 of the GFP has been substituted by G.

9. A fluorescent protein according to any one of the preceding claims having the amino acid sequence disclosed in SEQ ID NO: 4.

10. A fluorescent protein according to any one of the preceding claims having the amino acid sequence disclosed in SEQ ID NO: 8.

11. A fusion compound consisting of a fluorescent protein (GFP) according to any of the preceding claims, wherein the GFP is linked to a polypeptide.

12. A fusion compound according to the preceding claim, wherein the polypeptide is a kinase, preferably the catalytic subunit of protein kinase A, or protein kinase C, or Erk1, or a cytoskeletal element.

13. A nucleotide sequence coding for the Fluorescent protein of any of the preceding claims.

14. A nucleotide sequence according to the preceding claim, shown in SEQ ID NO: 3.

15. A nucleotide sequence according to the preceding claim, shown in SEQ ID NO: 7.

16. A nucleotide sequence according to any of the preceding claims in the form of a DNA sequence.

17. A host transformed with a DNA construct according to any of the preceding claims.

18. A process for preparing a polypeptide, comprising cultivating a host according to any of the preceding claims and obtaining therefrom the polypeptide expressed by said nucleotide sequence.

19. Use of the fluorescent protein according to any of the preceding claims in an in vitro assay for measuring protein kinase activity, or dephosphorylation activity, or for measuring protein redistribution.


SEQUENCE LISTING (24 sequences)

SEQ ID NO: 1 - DNA, 720 bp, Aequorea victoria
  CDS (1)..(717)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccacactagtgaccaccctgtcttacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - protein, 239 aa, Aequorea victoria
MVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 3 - DNA, 720 bp, Aequorea victoria
  CDS (1)..(717)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccacactagtgaccaccctgtcttacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctcctagggttcgtgaccgccgccgggatcactctcggcatggacgagctgtacaagtaa

SEQ ID NO: 4 - protein, 239 aa, Aequorea victoria
MVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLGFVTAAGITLGMDELYK

SEQ ID NO: 5 - DNA, 717 bp, Aequorea victoria
  CDS (1)..(714)
atgagtaaaggagaagaacttttcactggagttgtcccaattcttgttgaattagatggcgatgttaatgggcaaaaattctctgttagtggagagggtgaaggtgatgcaacatacggaaaacttacccttaaatttatttgcactactgggaagctacctgttccatggccaacgcttgtcactactctctcttatggtgttcaatgcttttctagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaattataactcacataatgtatacatcatggcagacaaaccaaagaatggcatcaaagttaacttcaaaattagacacaacattaaagatggaagcgttcaattagcagaccattatcaacaaaatactccaattggcgatggccctgtccttttaccagacaaccattacctgtccacgcaatctgccctttccaaagatcccaacgaaaagagagatcacatgatccttcttgagtttgtaacagctgctgggattacacatggcatggatgaagggtacaagtaa

SEQ ID NO: 6 - protein, 238 aa, Aequorea victoria
MSKGEELFTGVVPILVELDGDVNGQKFSVSGEGEGDATYGKLTLKFICTTGKLPVPWPTLVTTLSYGVQCFSRYPDHMKQHDFFKSAMPEGYVQERTIFYKDDGNYKTRAEVKFEGDTLVNRIELKGIDFKEDGNILGHKMEYNYNSHNVYIMADKPKNGIKVNFKIRHNIKDGSVQLADHYQQNTPIGDGPVLLPDNHYLSTQSALSKDPNEKRDHMILLEFVTAAGITHGMDEGYK

SEQ ID NO: 7 - DNA, 717 bp, Aequorea victoria
  CDS (1)..(717)
atgagtaaaggagaagaacttttcactggagttgtcccaattcttgttgaattagatggcgatgttaatgggcaaaaattctctgttagtggagagggtgaaggtgatgcaacatacggaaaacttacccttaaatttatttgcactactgggaagctacctgttccatggccaacgcttgtcactactctctcttatggtgttcaatgcttttctagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaattatacctcacataatgtatacatcatggcagacaaaccaaagaatggcatcaaagttaacttcaaaattagacacaacattaaagatggaagcgttcaattagcagaccattatcaacaaaatactccaattggcgatggccctgtccttttaccagacaaccattacctgtccacgcaatctgccctttccaaagatcccaacgaaaagagagatcacatgatcctcctagggtttgtaacagctgctgggattacacatggcatggatgaactatacaaataa

SEQ ID NO: 8 - protein, 238 aa, Aequorea victoria
MSKGEELFTGVVPILVELDGDVNGQKFSVSGEGEGDATYGKLTLKFICTTGKLPVPWPTLVTTLSYGVQCFSRYPDHMKQHDFFKSAMPEGYVQERTIFYKDDGNYKTRAEVKFEGDTLVNRIELKGIDFKEDGNILGHKMEYNYTSHNVYIMADKPKNGIKVNFKIRHNIKDGSVQLADHYQQNTPIGDGPVLLPDNHYLSTQSALSKDPNEKRDHMILLGFVTAAGITHGMDELYK

SEQ ID NO: 9 - DNA, 33 bp, Artificial Sequence
  Description of Artificial Sequence Primer
tgtactagtgaccaccctgtcttacggcgtgca

SEQ ID NO: 10 - DNA, 30 bp, Artificial Sequence
  Description of Artificial Sequence Primer
ctgactagtgtgggccagggcacgggcagc

SEQ ID NO: 11 - DNA, 42 bp, Artificial Sequence
  Description of Artificial Sequence Primer
cccggcggcggtcacgaaccctaggaggaccatgtgatcgcg

SEQ ID NO: 12 - DNA, 42 bp, Artificial Sequence
  Description of Artificial Sequence Primer
cgcgatcacatggtcctcctagggttcgtgaccgccgccggg

SEQ ID NO: 13 - DNA, 31 bp, Artificial Sequence
  Description of Artificial Sequence Primer
gtaccggtcaccatgagtaaaggagaagaac

SEQ ID NO: 14 - DNA, 29 bp, Artificial Sequence
  Description of Artificial Sequence Primer
ttattggtacccttcatccatgccatgtg

SEQ ID NO: 15 - DNA, 41 bp, Artificial Sequence
  Description of Artificial Sequence Primer
gagatcacatgatcctcctagggtttgtaacagctgctggg

SEQ ID NO: 16 - DNA, 41 bp, Artificial Sequence
  Description of Artificial Sequence Primer
cccagcagctgttacaaaccctaggaggatcatgtgatctc

SEQ ID NO: 17 - DNA, 34 bp, Artificial Sequence
  Description of Artificial Sequence Primer
ccaacgcttgtcacaacgttttcttatggtgttc

SEQ ID NO: 18 - DNA, 34 bp, Artificial Sequence
  Description of Artificial Sequence Primer
gaacaccataagaaaacgttgtgacaagcgttgg

SEQ ID NO: 19 - DNA, 34 bp, Artificial Sequence
  Description of Artificial Sequence Primer
cccacactagtgacaacgttttcttacggcgtgc

SEQ ID NO: 20 - DNA, 34 bp, Artificial Sequence
  Description of Artificial Sequence Primer
gcacgccgtaagaaaacgttgtcactagtgtggg

SEQ ID NO: 21 - DNA, 32 bp, Artificial Sequence
  Description of Artificial Sequence Primer
gttgtttcatgagtaaaggagaagaacttttc

SEQ ID NO: 22 - DNA, 31 bp, Artificial Sequence
  Description of Artificial Sequence Primer
gttggatccttatttgtatagttcatccatg

SEQ ID NO: 23 - DNA, 32 bp, Artificial Sequence
  Description of Artificial Sequence Primer
gttgttccatggtgagcaagggcgaggagctg

SEQ ID NO: 24 - DNA, 31 bp, Artificial Sequence
  Description of Artificial Sequence Primer
gttggatccttacttgtacagctcgtccatg